(19) United States
(12) Patent Application Publication
(10) Pub. No.: US 2023/0123485 A1
(43) Pub. Date: Apr. 20, 2023

(54) MICROBIAL COMPOSITIONS AND USES THEREOF

(71) Applicant: SUPERBREWED FOOD, INC., New Castle (DE)

(72) Inventors: Shawn William JONES, Bear, DE (US); Alon KARPOL, Tel-Mond (IL); Hadar GILARY, Magal (IL); Aharon M. EYAL, Jerusalem (IL); Bryan P. TRACY, Wilmington, DE (US)

(21) Appl. No.: 17/910,814

(22) PCT Filed: Mar. 3, 2021
(86) PCT No.: PCT/IB2021/051760

§ 371 Date: Sep. 11, 2022

Related U.S. Application Data
(60) Provisional application No. 62/988,054, filed on Mar. 11, 2020.

(51) Int. Cl. A61K 35/742; A61P 31/04; A23K 10/18

(52) U.S. Cl. CPC A61K 35/742; A61P 31/04; A23K 10/18

(57) ABSTRACT

Provided are microbial compositions comprising at least one attenuated form of a pathogenic bacterium having an ability to colonize at least one organ of the gastrointestinal tract of an animal, such as an attenuated form of a bacterium having an antagonistic effect against a bacterium selected from the group consisting of Salmonella enteritidis, Salmonella enterica, Salmonella typhimurium, lostridium difficile, Clostridium perfringens, Escherichia coli, Campylobacter spp., Listeria monocytogenes, Yersinia enterocolitica and combinations thereof. Also provided are animal feeds comprising such compositions, preparations for in-ovo injection comprising such compositions and methods of treatment using such compositions.

### CROSS-REFERENCE TO RELATED APPLICATION

The present application gains priority from U.S. Provisional Application No. 62/988,054 filed Mar. 11, 2020, which is incorporated by reference as if fully set-forth herein.

### FIELD OF THE INVENTION

The present invention, in at least some embodiments, relates to microbial compositions and in particular to microbial compositions comprising at least one attenuated form of a pathogenic bacterium having an ability to colonize the gastrointestinal tract of said animal and uses thereof in treating infection.

### BACKGROUND OF THE INVENTION

Infections may be caused by a variety of different microorganisms and may be transmitted in many different ways, such as by direct or indirect contact with an infected subject; via contaminated food or water; or via an insect bite.

Depending on the cause of the infection, treatment may involve administration of an anti-infective medication, such as antibiotic, antiviral or antifungal preparation. Some of these medications have been found to produce severe side effects in subjects receiving the treatment. Furthermore, certain infections have proven to be resistant to treatment.

There remains a need for an effective treatment for a wide range of different infections, which is devoid of at least some of the disadvantages of the prior art.

### SUMMARY OF THE INVENTION

According to an aspect of some embodiments of the present invention, there is provided a microbial composition for treating an infection in an animal, the composition comprising at least one attenuated form of a pathogenic bacterium having an ability to colonize at least one organ of the gastrointestinal tract of said animal, wherein said attenuated form of said pathogenic bacterium has an antagonistic effect against a bacterium selected from the group consisting of Salmonella enteritidis, Salmonella enterica, Salmonella typhimurium, Clostridium difficile, Clostridium perfringens, Escherichia coli, Campylobacter spp., Listeria monocytogenes, Yersinia enterocolitica and combinations thereof.

According to an aspect of some embodiments of the present invention, there is provided a feed comprising the composition as disclosed herein.

According to an aspect of some embodiments of the present invention, there is provided a preparation for in-ovo injection comprising the composition as disclosed herein.

According to an aspect of some embodiments of the present invention, there is provided a method for treating an infection in an animal, comprising administering to said animal the composition as disclosed herein.

### DETAILED DESCRIPTION OF THE INVENTION

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “microbial composition” refers to a composition comprising at least one live microorganism.

As used herein, the term “attenuated” refers to a bacterium which is virulent in its native form and which has been modified to remove or decrease the ability to infect or damage a host organism. As used herein, the term “decrease the ability to infect or damage” is intended to mean that the harmful effect of the bacterium on the host organism is decreased by at least 50%, at least 60%, at least 70%, at least 80%, at least 90% or by about 100% following modification as compared to the harmful effect of the native, unmodified bacterium.

As used herein, the term “treating” includes preventing, curing, ameliorating, mitigating, and reducing the instances or severity of a condition or a symptom thereof.

As used herein, the term “administering” includes any mode of administration, such as oral, subcutaneous, sublingual, transmucosal, parenteral, intravenous, intra-arterial, buccal, sublingual, topical, vaginal, rectal, ophthalmic, otic, nasal, inhaled, intramuscular, intraosseous, intrathecal, and transdermal, or combinations thereof. “Administering” can also include providing a different compound that when ingested or delivered as above will necessarily transform into the compound that is desired to be administered, this type of “different compound” is often being referred to as a “Prodrug”. “Administering” can also include prescribing or filling a prescription for a dosage form comprising a particular compound. “Administering” can also include providing directions to carry out a method involving a particular compound or a dosage form comprising the compound or compounds.

The amount of a attenuated form of a pathogenic bacterium to be administered is that which, when administered to a subject for treating an infection or a symptom thereof, is sufficient to have a beneficial effect with respect to that infection or symptom thereof. The amount to be administered will vary depending on the specific bacterium used, the specific infection or symptom thereof and its severity, and the age, weight, and other relevant characteristics of the subject to be treated. Determining the effective amount of a given attenuated form of a pathogenic bacterium to be administered is within the ordinary skill of the art and typically requires no more than routine experimentation.

As used herein, the term “colonize” with regard to bacteria means exist on a surface of a bodily tissue without causing disease in the host.

As used herein, the term “infection” refers to the presence of disease-causing microorganisms in a bodily tissue, such as bacteria, fungi, viruses and protozoa

As used herein, the term “free of infection” with regard to an animal refers to an animal having no delectable symptoms of an infection caused by microorganisms, when tested using standard methods as known in the art.

As used herein, the term “antagonistic effect” refers to a reduction of an effect of a native, virulent bacteria by an attenuated form of a pathogenic bacterium, wherein the native, virulent bacteria may be the same or different from the attenuated bacteria.

As used herein, the term “mutation” refers to an alteration of a nucleotide sequence of a gene. The alteration may comprise a deletion or replacement of a single nucleotide, an insertion (to generate frame-shift, or addition of a stop codon), or a missense (nonsense) mutation. Deletion can be achieved by mutation in the gene itself or in its regulation sites (such as the promoter, ribozyme binding site, repressor binding site, or termination site).

As used herein, the term “mutated toxin gene” refers to a toxin gene in which a mutation, as defined above, has occurred.

As used herein, the term “auxotrophic mutation” refers to a mutation of a strain that will block/prevent its ability to synthesize a chemical that is essential for growth.

As used herein, the term “membrane protein mutation” refers to mutation of a gene encoding a protein that is a part of, or interact with, a cell membrane resulting in an altered protein.

As used herein, the term “DNA recombination and repair mutation” refers to a mutation in a cell that is responsible for the repair of recombination events.

As used herein, the term “mutation of the hemolysin gene” refers to a hemolysin gene in which a mutation as defined above has occurred. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the present invention, provided is a microbial composition comprising at least one attenuated form of a pathogenic bacterium having an ability to colonize at least one organ of the gastrointestinal tract of an animal. According to some embodiments, said attenuated form of said pathogenic bacterium has an antagonistic effect against a bacterium selected from the group consisting of Salmonella enteritidis, Salmonella enterica, Salmonella typhimurium, Clostridium difficile, Clostridium perfringens, Escherichia coli, Campylobacter spp., Listeria monocytogenes, Yersinia enterocolitica and combinations thereof.

According to an embodiment, the bacterium has an ability to colonize at least one organ of the gastrointestinal tract selected from the group consisting of the mouth, esophagus, stomach, small intestine, large intestine and anus.

According to an embodiment, the microorganism colonizes at least 0.01%, at least 0.05%, at least 0.01%, at least 0.05%, at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80% or at least 90% of an organ of the gastrointestinal tract of the animal.

According to an embodiment, the microorganism colonizes two or more organs of the gastrointestinal tract, such as two sections, three sections, four sections, five or more organs.

The antagonistic effect may be achieved, for example, by secretion of molecules which prevent cell growth. The molecules may change the pH to unfavorable values, chelate essential nutrients, disrupt cell walls and cell membranes, interact with protein synthesis, or DNA replication, or disrupt any other essential function in the cell. Examples of such molecules include antibiotics, bacteriocins, toxins, and delivery devices.

According to an embodiment, said attenuated form of a pathogenic bacterium is characterized by having at least one selected from the group consisting of mutated toxin gene, auxotrophic mutation, membrane protein mutation, DNA recombination and repair mutation and combinations thereof.

According to an embodiment, said attenuated form of a pathogenic bacterium is an attenuated form of a strain of Clostridium perfringens having a mutation of the hemolysin gene.

According to an embodiment, the composition comprises attenuated forms of at least two, at least three, at least four at least five, at least six, at least seven, at least eight, at least nine or at least ten different genera of pathogenic bacteria.

According to an embodiment, the composition comprises attenuated forms of at least at least two, at least three, at least four at least five, at least six, at least seven, at least eight, at least nine or at least ten different species of pathogenic bacteria. In some such embodiments, the species are of same genus. In some such embodiments, the species are of different genera.

According to an embodiment, the microbial composition comprises between 10² and 10¹⁰ colony forming units of the attenuated form of a pathogenic bacterium, such as, for example, about 10², 10³, 10⁴, 10⁵, 10⁶, 10⁷, 10⁸, 10⁹, or 10¹⁰ colony forming units.

According to an embodiment, the microbial preparation comprises at least 10¹ Colony-Forming Units per milliliter of the microorganism. According to an embodiment, the microbial composition comprises at least 10¹, at least 10², at least 10³, at least 10⁴, at least 10⁵, at least 10⁶, at least 10⁷, at least 10⁸, at least 10⁹ CFU per millimeter of the microorganism. According to an embodiment, the microbial composition comprises up to 10¹⁰ CFU per millimeter of the microorganism.

According to an embodiment, the microbial composition further comprises at least one selected from the group consisting of water, macronutrients, prebiotics, and probiotics and combinations thereof.

According to an embodiment, the macronutrients are selected from the group consisting of carbohydrates (such as glycogen, starch, cellulose, cellobiose, sucrose, maltose, lactose, mannose, galactose, fructose, inulin, fructooligosachharides, Galactooligosaccharide, mannan oligosaccharide, ribose, Trehalose, dextrose, multidextrin, pectin and combinations thereof); fats (such as saturated fat, unsaturated fat, monounsatutrated fat, polyunsaturated fat, trans fat, triglycerides, cholesterol, fatty acids, and combinations thereof); proteins (such as free amino acids, peptides, and combinations thereof); and combinations thereof.

According to an embodiment, the microorganism is present as a live vegetative culture.

According to an embodiment, the microorganism is present as a sporulated culture.

According to an embodiment, the microbial composition further comprises a carrier selected from the group consisting of water, saline, aqueous dextrose, lactose, a buffered solution, starch, cellulose, glucose, lactose, sucrose, gelatin, malt, rice, flour and combinations thereof.

According to an embodiment, the composition further comprises at least one organism selected from the group consisting of an acetogenic bacterium, a butyric acid producing bacterium, a lactic acid consuming bacterium and combinations thereof.

According to an aspect of some embodiments of the present invention, titer is provided a feed comprising the composition as disclosed herein.

According to an aspect of some embodiments of the present invention, there is provided a preparation for comprising the composition as disclosed herein for use in therapy by in ovo injection.

According to an aspect of some embodiments of the present invention, there is provided a method for treating an infection in an animal, comprising administering to said animal the composition as disclosed herein.

According to an aspect of some embodiments of the present invention, the composition is for use in treating an infection in an animal.

According to an embodiment, said infection is caused by a bacterium selected from the group consisting of Salmonella enteritidis, Salmonella enterica, Salmonella typhimurium, Clostridium difficile, Clostridium perfringens, Escherichia coli, Campylobacter spp., Listeria monocytogenes, Yersinia enterocolitica and combinations thereof.

According to an embodiment, said infection is selected from the group consisting of Salmonella enteritidis, Salmonella enterica, Salmonella typhimurium, Clostridium difficile, Clostridium perfringens, Escherichia coli, Campylobacter spp., Listeria monocytogenes, Yersinia enterocolitica and combinations thereof.

According to an embodiment, said infection is selected from the group consisting of Campylobacteriosis, Salmonellosis, Necrotic enteritis, Colibacillosis, Listeriosis, and Yersiniosis.

According to an embodiment, said attenuated form of a pathogenic bacterium is a attenuated form of a bacterium causing said infection. In some such embodiments, competition for a food source occurs between the attenuated and native forms of the bacteria. In some such embodiments, when said attenuated form of a pathogenic bacteria are administered to an animal which is free of infection, an immune response is elicited by the attenuated form which provides protection also against the native form. In some such embodiments, the immune response is an innate immune response.

According to an embodiment, said attenuated form of a pathogenic bacterium is an attenuated form of a bacterium other than the bacterium causing said infection. In some such embodiments, said attenuated form of a pathogenic bacterium generates a specific antibiotic against the bacteria causing said infection. In some such embodiments, the attenuated form of a pathogenic bacterium is capable of colonizing the gastrointestinal tract at a faster rate than the bacteria causing said infection.

According to an embodiment, administering is selected from the group consisting of feeding and in-ovo injection.

According to an embodiment, said animal is free of said infection prior to said administering.

According to an embodiment, said administering is to an infected animal.

According to an embodiment, said animal is selected from the group consisting of avians, mammals, swine, ruminants, fish, Crustacea, and reptiles.

According to an embodiment, the microbial composition, feed or preparation is for use by administration in the absence of an antibiotic.

According to an embodiment, said treating is devoid of administration of an antibiotic.

According to an embodiment, antibiotics are administered in addition to treating h the composition of the present invention.

According to an embodiment, the microbial composition dose is administered at least twice.

According to an embodiment, the microbial composition is administered once a day, twice a day, three times a day or more for a given period.

According to an embodiment, administering once a day comprises administering in the morning. According to an embodiment, administering once a day comprises administering in the afternoon. According to an embodiment, administering once a day comprises administering in the evening.

According to an embodiment, administering twice a day comprises administering in the morning and the afternoon. According to an embodiment, administering twice a day comprises administering in the morning and the evening. According to an embodiment, administering twice a day comprises administering in the afternoon and the evening.

According to an embodiment, administering three times a day comprises administering in the morning, afternoon and evening.

According to an embodiment, administering is carried out once, twice, three times or more per day for one day, two days, three days, four days, five days, six days, one week, two weeks, three weeks, one month, two months, three months, four months, five months, six months, seven months, eight months, nine months, ten months, eleven months, one year or more than one year.

According to an embodiment, administering is carried out by a route selected from the group consisting of enteral (such as oral, sublingual, buccal, gastric, duodenal or rectal) or parenteral (such as IV, intramuscular subcutaneous and transdermal routes).

According to an embodiment, administering is carried out by oral administration. According to some such embodiments, the composition is provided in the form of a solid oral dosage form, such as a tablet, capsule, sachet, powder, granule. According to some such embodiments, the composition is provided in the form of a liquid oral dosage form, such as a syrup, solution or dispersion. According to some embodiments, the composition further comprises one or more excipients, such as sweeteners, colorants, flavorants, stabilizers and the like.

According to an embodiment, the effective dose is configured to provide at least 10 organism Colony-Forming Units per gram wet feces of the animal on a second day after administering the composition.

According to an embodiment, the colonizing is configured to provide at least 100 Colony-Forming Units per gram wet feces of the animal on a twentieth day after administering the composition.

### EXAMPLES

### Example 1

### Composition Comprising Clostridiium perfingens for Treatment of Chickens

Clostridium perfringens is known to be a major cause of enteritis (NE) in chickens.

A Clostridium perfringens strain is isolated from chicken ceca. This strain has the ability to inhibit the growth of Salmonella and E. Coli. However, it also has toxin A and toxin B genes which are virulent. These genes are removed by standard methods known in the field of molecular biology, thereby removing the pathogenicity of the bacteria to provide an attenuated form of the bacteria. The attenuated bacteria are then added to feeds of test groups as a preventive measure to colonize the gut and prevent the colonization of pathogenic Clostridium perfringens, as well as to protect against Salmonella and E. Coli.

The bacteria are grown under anaerobic conditions. 120 chicks are divided into 2 groups of 60 each:

Group I: control group—no Clostridium perfringens administered;

Group II: attenuated Clostridium perfringens are added to feed at concentration of 10⁷ colony forming units/Kg of feed for the first week after hatching.

After the first week, each group is subdivided into 2 subgroups of 30 each, and one subgroup is challenged with pathogenic Clostridium perfringens while a second subgroup is challenged with pathogenic Salmonella.

Mortality and health of the chicks is examined over the subsequent 2 weeks.

Results show that chicks that are given Clostridium perfringens during the first week of their lives show a reduction in infection by either C. perfringens (40% reduction) or Salmonella (25% reduction) compared to birds that do not receive the attenuated bacteria in the feed.

### Example 2

### Composition Comprising Clostridiium difficile for Treatment of Piglets

Clostridium difficile infection commonly occurs in swine mostly resulting in diarrhea. In piglets usually up to 1 week of age, infection may cause enteritis and prove to be fatal.

A Clostridium difficile strain is isolated from pig colon. The strain comprises two virulent exotoxins A and B (TcdA and TcdB). These genes are removed by standard methods known in the field of molecular biology, thereby removing the pathogenicity of the bacteria to provide an attenuated form of the bacteria.

The attenuated bacteria are given to pregnant pigs before birth to ensure exposure to non-pathogenic Clostridium difficile that compete with the pathogenic strain. The bacteria are grown under anaerobic conditions.

Attenuated bacteria are added to the feed of a pregnant pig at a concentration of 10⁷ colony forming units/Kg of feed, during the last month of pregnancy and onwards. On day 5 after birth, the litter from the pig fed with attenuated bacteria and a litter of a pig that is not fed the attenuated bacteria during pregnancy (control pig) are each challenged with pathogenic Clostridium difficile. The health of the piglets is examined during the following week.

Results showed that addition of the attenuated bacteria to the mother's diet protects the piglets against infection by Clostridium difficile. Only 20% of the litter from the pig fed with Clostridium difficile have diarrhea whereas the from the control pig litter have a 75% infection rate.

1. A microbial composition comprising at least one attenuated form of a pathogenic bacterium having an ability to colonize at least one organ of the gastrointestinal tract of said animal, wherein said attenuated form of said pathogenic bacterium has an antagonistic effect against a bacterium selected from the group consisting of Salmonella enteritidis, Salmonella enterica, Salmonella typhimurium, Clostridium difficile, Clostridium perfringens, Escherichia coli, Campylobacter spp., Listeria monocytogenes, Yersinia enterocolitica and combinations thereof.

2. The microbial composition of claim 1, wherein said attenuated form of a pathogenic bacterium has at least one modification selected from the group consisting of mutated toxin gene, auxotrophic mutation, membrane protein mutation, DNA recombination and repair mutation and combinations thereof.

3. The microbial composition of claim 1, wherein said attenuated form of a pathogenic bacterium is an attenuated form of a strain of Clostridium perfringens having a mutation of the hemolysin gene.

4. The microbial composition of claim 1, further comprising a carrier selected from the group consisting of water, saline, aqueous dextrose, lactose, a buffered solution, starch, cellulose, glucose, lactose, sucrose, gelatin, malt, rice, flour and combinations thereof.

5. The microbial composition of claim 1, further comprising at least one organism selected from the group consisting of an acetogenic bacterium, a butyric acid producing bacterium, a lactic acid consuming bacterium and combinations thereof.

6. A feed comprising the composition of claim 1.

7-15. (canceled)

16. A method for treating or preventing an infection in an animal, comprising administering to said animal the composition of claim 1.

17. The method of claim 16, wherein said infection is selected from the group consisting of Campylobacteriosis, Salmonellosis, Necrotic enteritis, Colibacillosis, Listeriosis, and Yersiniosis.

18. The method of claim 16, wherein said attenuated form of a pathogenic bacterium is an attenuated form of a bacterium causing said infection.

19. The method of claim 16, wherein said attenuated form of a pathogenic bacterium is an attenuated form of a bacterium other than a bacterium causing said infection.

20. The method of claim 16, wherein said administering is selected from feeding and in-ovo injection.

21. The method of claim 16, for preventing said infection, wherein said animal is free of said infection prior to said administering.

22. The method of claim 16, for treating an animal, wherein said administering is to an infected animal.

23. The method of claim 16, wherein said animal is selected from the group consisting of Avian, mammals, swine, ruminants, fish, Crustacea, and reptiles.

24. The method of claim 16, wherein said treating is devoid of administration of antibiotics.

25. The method of claim 16, wherein said bacterium causing said infection is selected from the group consisting of Salmonella enteritidis, Salmonella enterica, Salmonella typhimurium, Clostridium difficile, Clostridium perfringens, Escherichia coli, Campylobacter spp., Listeria monocytogenes, Yersinia enterocolitica and combinations thereof.
